(19) United States
(12) Patent Application Publication
(10) Pub. No.: US 2022/0287716 A1
(43) Pub. Date: Sep. 15, 2022

(54) CIRCULAR STAPLING DEVICE FOR END-TO-END ANASTOMOSIS

(71) Applicant: Covidien LP, Mansfield, MA (US)

(72) Inventor: Olesea Diaz-Chiosa, Naugatuck, CT (US)

(73) Assignee: Covidien LP, Mansfield, MA (US)

(21) Appl. No.: 17/679,430

(22) Filed: Feb. 24, 2022

Related U.S. Application Data
(60) Provisional application No. 63/159,529, filed on Mar. 11, 2021.

(51) Int. Cl. A61B 17/115

(52) U.S. Cl. CPC A61B 17/1155; A61B 2090/0807

(57) ABSTRACT

A circular stapling device for performing anastomosis procedures includes an anvil assembly and a reload assembly that supports a staple cartridge. The anvil assembly has an anvil head that includes an anvil surface with staple forming pockets that are coated with a plurality of layers of materials having different colors. The colors that are exposed after the stapling device is fired provides an indication to a clinician of whether tissue clamped between the anvil assembly and the reload assembly was clamped in optimal fashion.

### CROSS-REFERENCE TO RELATED APPLICATION

This application claims the benefit of and priority to U.S. Provisional Patent Application No. 63/159,529, filed Mar. 11, 2021, the entire contents of which is incorporated by reference herein.

### FIELD

This technology is generally related to surgical stapling devices and, more particularly, to a surgical stapling device for performing end-to-end anastomosis that has structure on an anvil assembly to indicate to a clinician if tissue was gripped consistently about the anastomotic site during an anastomosis procedure.

### BACKGROUND

Circular stapling devices are commonly used to perform anastomoses during surgical procedures. Anastomosis is the surgical connection of two body lumens, e.g., blood vessels, intestine, etc. Leaks in an anastomosis can lead to bleeding and result in infection which may result in life-threatening complications. As such, it is common practice for a clinician to examine an anastomotic donut formed during the anastomosis procedure after the anastomosis procedure is completed to verify that there are no leaks or bleeding. Examination of the anastomotic donut typically requires an endoscopy procedure which is performed after the anastomosis procedure has been performed. This adds cost and time to the anastomosis procedure.

During a colectomy procedure, a portion of the gastrointestinal (GI) tract is resected and GI tract sections on opposite sides of the resected tract section are rejoined to form the anastomotic donut. This procedure is conducted within the GI tract and is performed without visualization of the anastomotic site. As such, a clinician, e.g., a surgeon, typically inspects the anastomotic donut after the anastomosis procedure is completed. This inspection requires preparing a colonoscope and visualization capabilities and poking an end of the colonoscope into the freshly resected and stapled tract sections to determine if the anastomotic donut is properly formed and free from leaks. If the anastomotic donut is not properly formed, the inspection risks perforation of the anastomosis. In addition to the risk of perforation, the inspection increases operating time and requires significant surgical skill and staff coordination to complete.

A continuing need exists in the art for a safer, less time-consuming way to evaluate the effectiveness of the anastomosis after an anastomosis procedure has been performed.

### SUMMARY

This disclosure is directed to a circular stapling device for performing anastomosis procedures that includes an anvil assembly and a reload assembly that supports a staple cartridge. The anvil assembly has an anvil head that includes an anvil surface with staple forming pockets that are coated with a plurality of layers of materials having different colors. The colors that are exposed after the stapling device is fired provides an indication to a clinician of whether tissue clamped between the anvil assembly and the reload assembly was clamped in an optimal fashion or in a sub-optimal fashion.

Aspects of this disclosure are directed to an anvil assembly including an anvil shaft, an anvil head, and a plurality of layers of coating material. The anvil shaft has a proximal portion and a distal portion. The anvil head has an anvil surface that defines staple forming pockets. The plurality of layers of coating material is positioned on the anvil surface within the staple forming pockets and includes an inner layer having a first color and an outer layer having a second color that is different from the first color. The outer layer is positioned over the inner layer.

Another aspect of the disclosure is directed to an anvil assembly that includes an anvil shaft, an anvil head, and a plurality of layers of coating material. The anvil shaft has a proximal portion and a distal portion. The anvil head has an anvil surface that defines staple forming pockets. The plurality of layers of coating material is positioned on the anvil surface within the staple forming pockets. Each of the plurality of layers of coating material is formed of color coated metal. The plurality of layers of coating material includes an inner layer having a first color, a middle layer having a second color, and an outer layer having a third color. Each of the first, second, and third colors is different from the others.

In aspects of the disclosure, the plurality of layers of coating material includes a middle layer that has third color that is different than the first and second colors and is positioned between the inner and outer layers.

In some aspects of the disclosure, the plurality of layers of coating material are formed from color coated metals.

In certain aspects of the disclosure, the anvil head includes a central post that is secured to the proximal portion of the anvil shaft by a pivot member.

In aspects of the disclosure, the proximal portion of the anvil shaft is configured to be releasably coupled to a surgical stapling device.

In some aspects of the disclosure, the plurality of layers of coating material is applied to the anvil surface within the staple forming pockets by vapor deposition.

In certain aspects of the disclosure, inner layer has a thickness of from about 0.3 mm to about 0.5 mm, the middle layer has a thickness of from about 0.3 mm to about 0.5 mm, and outer layer has a thickness of from about 0.3 mm to about 0.5 mm.

In aspects of the disclosure, each of the staple forming pockets includes first and second pocket portions and a linear portion, and the linear portion extends between and interconnects the first and second pocket portions.

Other aspects of the disclosure are directed to a surgical stapling device that includes an elongate body, a reload assembly, and an anvil assembly. The elongate body has a proximal portion and a distal portion. The reload assembly is supported on the distal portion of the elongate body and includes a shell housing, a staple cartridge, staples supported within the staple cartridge, and a pusher. The pusher is movable within the shell housing from a retracted position to an advanced position to eject the staples from the staple cartridge. The anvil assembly includes an anvil shaft, an anvil head, and a plurality of layers of coating material. The anvil shaft has a proximal portion and a distal portion. The anvil head has an anvil surface that defines staple forming pockets. The plurality of layers of coating material are positioned on the anvil surface within the staple forming pockets and includes an inner layer having a first color and an outer layer having a second color that is different from the first color. The outer layer is positioned over the inner layer.

In aspects of the disclosure, the reload assembly includes a cutting blade that is supported on the pusher.

In some aspects of the disclosure, a handle assembly is coupled to the proximal portion of the elongate body.

Other features of the disclosure will be appreciated from the following description.

### BRIEF DESCRIPTION OF THE DRAWINGS

Various aspects of the disclosure are described herein below with reference to the drawings, wherein:

FIG. 1 is a side perspective view of a surgical stapling device according to aspects of the disclosure with an anvil assembly of the stapling device separated from an elongate body of the surgical stapling device;

FIG. 2 is a side perspective view from the proximal end of the anvil assembly shown in FIG. 1;

FIG. 3 is a cross-sectional view taken along section line 3-3 of FIG. 2;

FIG. 4 is a side view of a distal portion of the surgical stapling device shown in FIG. 1 with the distal portion of the surgical stapling device shown in phantom and tract sections of a body lumen clamped between the anvil assembly and a shell assembly of the surgical stapling device;

FIG. 5 is a cross-sectional view taken along section line 5-5 of FIG. 4 with the tract sections clamped between the anvil assembly and the shell assembly in optimal fashion;

FIG. 6 is a cross-sectional view taken through a portion of the anvil assembly including a staple forming pocket and a side view of a staple formed in the staple forming pocket through tissue clamped in optimal fashion;

FIG. 7 is a side perspective view from the proximal end of the distal portion of the anvil assembly after the surgical stapling device has been fired with the tract sections clamped between the anvil assembly and the shell assembly in optimal fashion;

FIG. 8 is a cross-sectional view of an anastomotic donut formed between the tract sections with the tract sections clamped between the anvil assembly and the shell assembly in optimal fashion;

FIG. 9 is a cross-sectional view taken along section line 5-5 of FIG. 4 with the tract sections clamped between the anvil assembly and the shell assembly in sub-optimal fashion;

FIG. 9A is a cross-sectional view taken through a portion of the anvil assembly including a staple forming pocket and a side view of a staple formed in the staple forming pocket through tissue clamped in sub-optimal fashion;

FIG. 10 is a side perspective view from the proximal end of the distal portion of the anvil assembly after the surgical stapling device has been fired with the tract sections clamped between the anvil assembly and the shell assembly in sub-optimal fashion; and

FIG. 11 is a cross-sectional view of an anastomotic donut formed between the tract sections with the tract sections clamped between the anvil assembly and the shell assembly in sub-optimal fashion.

### DETAILED DESCRIPTION

The disclosed circular stapling device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including surgeons, doctors, nurses, and support personnel. In addition, directional terms such as front, rear, upper, lower, top, bottom, and similar terms are used to assist in understanding the description and are not intended to limit the disclosure.

This disclosure is directed to a circular stapling device for performing anastomosis procedures that includes an anvil assembly and a shell assembly that supports a staple cartridge. The anvil assembly has an anvil head that includes an anvil surface with staple forming pockets that are coated with a plurality of layers of materials having different colors. The staple cartridge supports a plurality of staples. When the stapling device is fired, legs of staples are received and formed within the staple forming pockets of the anvil surface and penetrate one or more of the layers of materials. The trajectory of the staple legs as the staple legs are formed is affected by a resistance of tissue clamped between the staple cartridge and the anvil plate. More specifically, the amount of tissue and the type of tissue clamped between the staple cartridge and the anvil plate will affect the trajectory of the staple legs as the staple legs are advanced through the tissue and are formed within the staple forming pockets. If a large amount of tissue and/or tissue having a high tensile strength is clamped between the staple cartridge and the anvil plate, the staple legs will take a shallow trajectory through the layer or layers of materials coating the staple forming pockets as the staple legs are formed. Conversely, if a lesser amount of tissue and/or tissue having a low tensile strength is clamped between the staple cartridge and the anvil plate, the staple legs will take a deep trajectory through the layers of materials coating the staple forming pockets. After a surgical procedure, a clinician can inspect the exposed colored layers of coating within the staple forming pockets of the anvil plate to determine whether an adequate amount of tissue and/or the correct type of tissue was clamped between the staple cartridge and the anvil plate to form an anastomosis. Based on the results of this inspection, a need for inspection of the anastomosis using a colonoscope may be obviated.

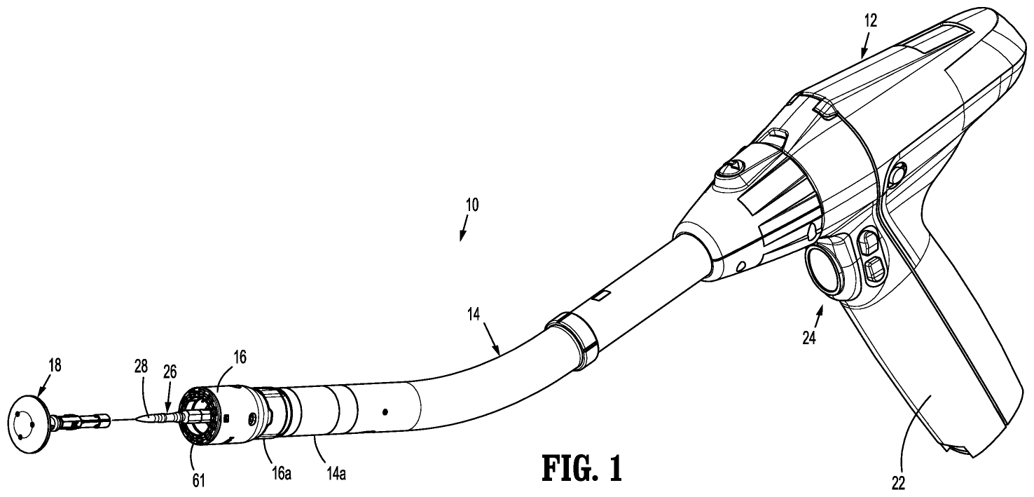

FIG. 1 illustrates a circular stapling device according to aspects of the disclosure shown generally as stapling device 10. The stapling device 10 includes a handle assembly 12, an elongate body or adaptor assembly 14, a reload assembly 16, and an anvil assembly 18. The anvil assembly 18 is releasably supported on a distal end of the adaptor assembly 14 and is movable in relation to the reload assembly 16 between spaced and clamped positions as is known in the art. The reload assembly 16 includes a proximal portion 16a that is releasably coupled to a distal portion 14a of the elongate body 14. Alternately, the reload assembly 16 can be fixedly secured to the distal portion 14a of the elongate body 14. In certain aspects of the disclosure, the handle assembly 12 includes a stationary grip 22 that supports actuation buttons 24 for controlling operation of various functions of the stapling device 10 including approximation of the reload and anvil assemblies 16 and 18, respectively, firing of staples from the reload assembly 16, and cutting or coring of tissue. The elongate body 14 supports an anvil retainer 26 that includes a distal trocar portion 28. The trocar portion 28 includes an annular shoulder 28a.

The stapling device 10 is illustrated as a powered stapling device and includes an electrically powered handle assembly 12 that may support one or more batteries (not shown). The elongate body 14 is in the form of an adaptor assembly that translates power from the handle assembly 12 to the reload and anvil assemblies 16 and 18, respectively. Examples of electrically powered stapling devices can be found in U.S. Pat. Nos. 9,055,943, 9,023,014, and U.S. Publication Nos. 2018/0125495, and 2017/0340351. Alternately, it is envisioned that the stapling device 10 could also be a manually powered stapling device such as disclosed in U.S. Pat. No. 7,303,106 (the '106 patent) or a stapling device that is configured for use with a robotic system such as disclosed in U.S. Pat. No. 9,962,159 that does not include a handle assembly.

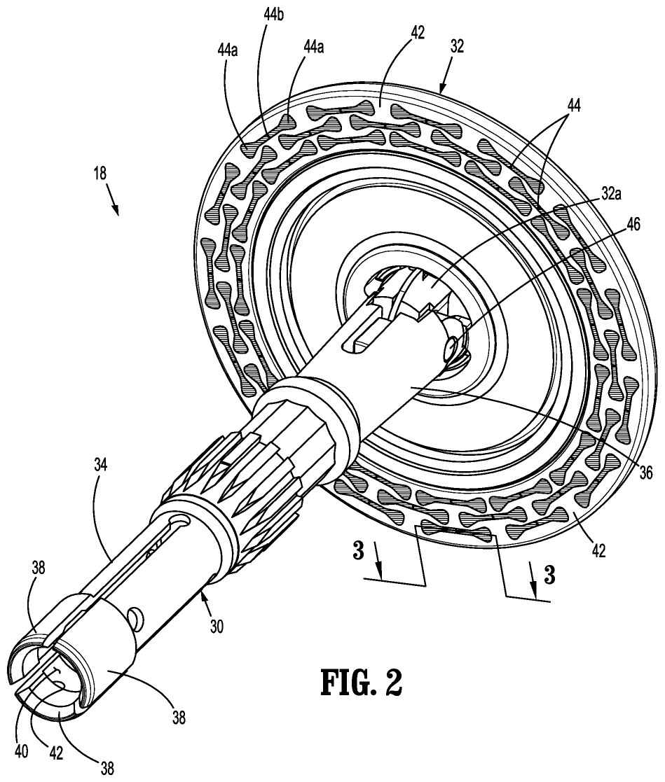

FIG. 2 illustrates the anvil assembly 18 which includes an anvil shaft 30 and an anvil head 32. The anvil shaft 30 includes a proximal portion 34 and a distal portion 36. The proximal portion 34 of the anvil shaft 30 includes a plurality of flexible legs 38 that define a channel 40 that receives the anvil retainer 26 to releasably secure the anvil assembly 18 to the anvil retainer 26. In aspects of the disclosure, the anvil shaft 30 includes an inner shoulder 42 that engages the annular shoulder 28a (FIG. 1) on the anvil retainer 26 to secure the anvil assembly 18 to the distal trocar portion 28 of the anvil retainer 26. For a more detailed description of the releasable connection between the anvil assembly 18 and the anvil retainer 26, see the '106 patent.

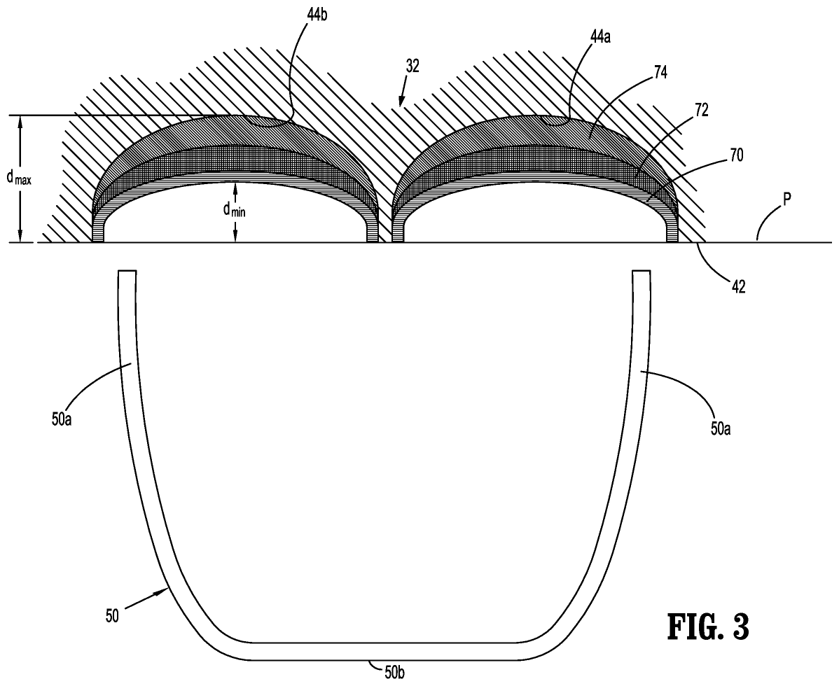

The anvil head 32 includes a central post 32a and an annular anvil surface 42 that faces the reload assembly 16 (FIG. 1) and is positioned about the central post 32a. The annular anvil surface 42 defines a plurality of staple forming pockets 44. In aspects of the disclosure, the staple forming pockets 44 are aligned in one or more annular rows formed about the central post 32a. In some aspects of the disclosure, each of the staple forming pockets 44 includes first and second pocket portions 44a that are interconnected by a linear portion 44b. The first and second pocket portions 44a are defined by curved walls that are configured to form legs 50a of a staple 50 (FIG. 3) into a B-shaped configuration. The linear portions 44b of the staple forming pockets 44 include side walls that confine the legs 50a of the staples 50 as the staple legs 50a are formed to align the legs 50a of the staples 50 with each other. Alternately, other staple forming pocket configurations are envisioned. In aspects of the disclosure, the central post 32a of the anvil head 32 is coupled to the distal portion 36 of the anvil shaft 30 by a pivot member 46 to allow the anvil head 32 to pivot in relation to the anvil shaft 30 from an operative position (FIG. 2) to a tilted position. It is also envisioned that the anvil head 32 can be fixedly secured to the anvil shaft 30 in the operative position.

FIG. 3 illustrates the staple 50 which includes the legs 50a and a back span 50b (FIG. 3) that connects the legs 50a to each other. In aspects of the disclosure, the legs 50a have a proximal end that is integrally formed with the back span 50b and a distal end that is configured to penetrate tissue. Although the distal ends of the legs 50a are shown to be flat, it is envisioned that the distal ends of the legs 50a can be tapered to penetrate tissue more easily.

FIG. 5 illustrates a distal portion of the reload assembly 16 which includes a shell housing 60 that supports an annular staple cartridge 61, a pusher 64, and a cutting blade 65. The staple cartridge 61 defines staple receiving slots 62. Each of the staple receiving slots 62 receives one of the staples 50. The pusher 64 includes fingers 66 that are received in each of the staple receiving slots 62 and is engaged with the back span 50b (FIG. 3) of one of the staples 50. The cutting blade 65 has an annular configuration and is supported about an inner periphery of the pusher 64. The pusher 64 is movable within the shell housing 60 from a retracted position to an advanced position to drive the staples 50 from the staple cartridge 61 into the anvil surface 42 of the anvil head 32 and to advance the cutting blade 65 through the staple cartridge 61. For a more detailed description of an exemplary shell assembly, see the '106 patent.

FIG. 3 illustrates an enlarged view of one of the staple forming pockets 44 of the anvil head 32. Each of the walls defining the staple forming pockets 44 is coated with layers of a colored material, e.g., a color coated metal. Each layer of colored material is a different color than the other layers of colored material and different from the surrounding material used to form the anvil head 32, e.g., stainless steel. In aspects of the disclosure, the layers of colored materials include a first or outer layer 70, a second middle layer 72, and a third inner layer 74. The first layer 70 has a first color, e.g., blue, the second layer 72 has a second color, e.g., yellow, and the third layer 74 has a third color, e.g., green. The third or inner layer 74 is positioned on the anvil surface 44 within the staple forming pockets 44. The first layer 70 is positioned on top of the second layer 72, and the second layer 72 is positioned on top of the third layer 74. Although three layers of colored material are shown, it is envisioned that the staple pockets 44 could be coated with two or more layers of colored material.

The colored materials forming the layers 70, 72, and 74 are applied to the walls defining the staple forming pockets 44 in a manner to facilitate removal of one or more of the layers 70, 72, and 74 from the staple forming pockets 44 upon engagement with the distal ends of the staple legs 50a of the staples 50. The colored materials of each of the layers 70, 72, and 74 can be applied to the walls defining the staple forming pockets 44 using a variety of different techniques including brushing, vapor deposition, or the like. In aspects of the disclosure, layer 70 has a thickness of from about 0.3 mm to about 0.5 mm, layer 72 has a thickness of from about 0.3 mm to about 0.5 mm, and layer 74 has a thickness of from about 0.3 mm to about 0.5 mm. In some aspects of the disclosure, one or more of the layers 70, 72, and 74 can be scratched off the walls defining the staple forming pockets 44 by engagement with the distal ends of the staple legs 50a upon firing of the stapling device 10 (FIG. 1).

In aspects of the disclosure, the anvil surface 42 of the anvil head 32 defines a plane “P” and each of the layers 70, 72, and 74 of colored material is positioned within the staple forming pocket 44 at a different distance from the plane “P”. The first layer 70 has a proximal-most surface that has the shallowest depth “dₘᵢₙ” within the staple forming pocket 44, the anvil surface 42 upon which the third layer 74 of colored material is applied defines the deepest depth “dₘₐₓ” within each of the staple forming pockets 44, and the second and third layers 72 and 74 of colored materials have proximal-most surfaces that are positioned at different depths within the staple forming pocket 44 between “dₘₐₓ” and “dₘᵢₙ”.

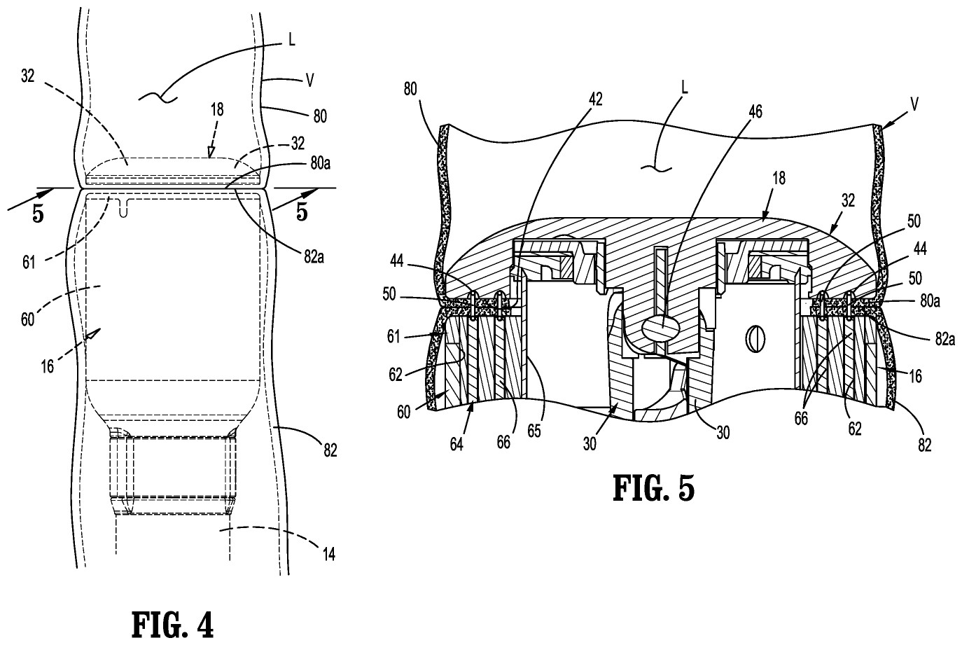

FIGS. 4 and 5 illustrate the distal portion of the stapling device 10 with the stapling device 10 in a clamped position. As summarized above, the anvil assembly 18 is releasably supported on the anvil retainer 26 (FIG. 1) and is movable with the anvil retainer 26 in relation to the staple cartridge 61 of the shell assembly 16 between spaced and clamped positions. In the clamped position, the anvil surface 42 of the anvil head 32 and a distal surface of the staple cartridge 61 are in juxtaposed alignment to clamp tissue therebetween. When the stapling device 10 is fired to eject the staples 50 from the staple receiving slots 62 of the staple cartridge 61, the distal ends of the staple legs 50a (FIG. 3) of each of the staples 50 exit the staple cartridge 61, pass through tissue clamped between the staple cartridge 61 and the anvil surface 42 and enter the respective first and second pocket portions 44a of the staple forming pockets 44 where the staple legs are formed into a B-shape.

The resistance exerted on the staple legs 50a by the tissue clamped between the staple cartridge 61 and the anvil surface 42 will affect the trajectory of formation of the staple legs 50a within each of the staple forming pockets 44 of the anvil head 32 of the anvil assembly 18. More specifically, the amount of tissue and the type of tissue clamped between the staple cartridge 61 and the anvil surface 42 of the anvil head 32 will affect the trajectory of formation of the staple legs 50a as the staple legs 50a are advanced through the tissue and formed within the staple forming pockets 44. If a large amount of tissue and/or tissue having a high tensile strength is clamped between the staple cartridge 61 and the anvil surface 42, the staple legs 50a will take a shallow trajectory through the layer or layers of materials 70, 72, and 74 coating the staple forming pockets 44 as the staple legs 50a are formed. Conversely, if a lesser amount of tissue and/or tissue having a low tensile strength is clamped between the staple cartridge 61 and the anvil surface 42, the staple legs 50a will take a deep trajectory through the layers of materials 70, 72, and 74 coating the staple forming pockets 44.

During an anastomosis procedure, first and second tracts 80 and 82 of a vessel “V”, e.g., the colon, are reconnected after a diseased portion (not shown) of the vessel “V” has been resected. As shown, the stapling device 10 is inserted into a lumen “L” defined by the two tracts 80 and 82 with the anvil head 32 of the anvil assembly 18 positioned in the first tract vessel 80 and the reload assembly 16 positioned in the second tract 82 of the vessel “V”. The anvil head 32 is approximated towards the shell assembly 16 to clamp ends 80a and 82a of the two tracts 80 and 82 between the anvil surface 42 of the anvil head 32 and the staple cartridge 61 of the reload assembly 16. When the anvil head 32 of the anvil assembly 18 and the staple cartridge 61 of the reload assembly 16 are in a clamped position, each of the staple forming pockets 44 of the anvil head 32 is aligned with a respective one of the staple receiving slots 62 of the staple cartridge 61 such that the staples 50 ejected from the staple cartridge 61 are formed into a substantially B-shape through both ends 80a and 80b of the first and second vessel tracts 80 and 82, respectively.

The colon is comprised of four types of tissue including mucosa, submucosa, muscularis, and serosa. For optimal anastomotic integrity, the muscularis and submucosal tissue of both first and second vessel tracts 80 and 82 must be clamped or gripped between the staple cartridge 61 of the reload assembly 16 and the anvil surface 42 such that the staples 50 are forced through these tissues. This is necessary because the muscularis and submucosa have the greatest tensile strength and are less resistant to tearing. Where only mucosal tissue is clamped between the staple cartridge 61 of the reload assembly 16 and the anvil surface 42, an anastomotic donut 90 (FIG. 8) formed during the procedure is more prone to leakage. Thus, it is important that muscularis and submucosa tissue from both ends 80a and 82a of the first and second vessel tracts 80 and 82 are properly clamped between the staple cartridge 61 and the anvil surface 42 and form part of the anastomotic donut 90 (FIG. 8).

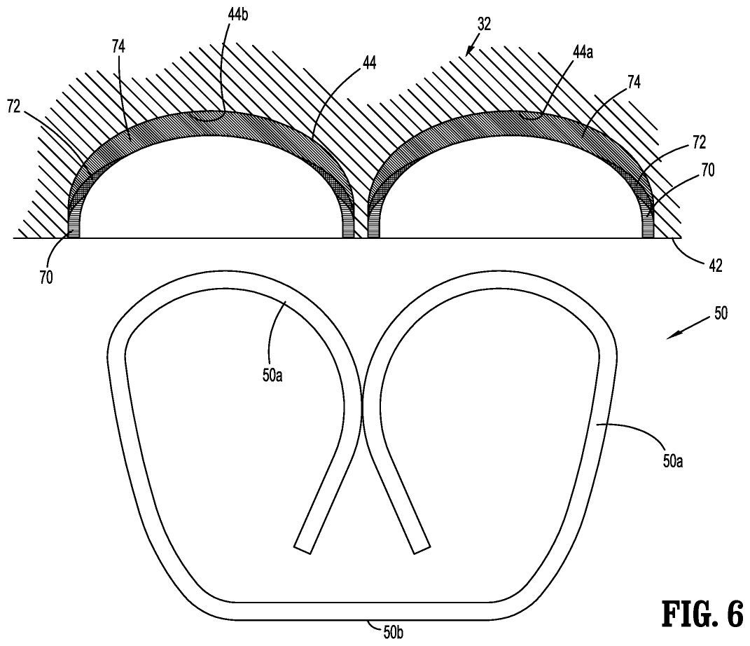

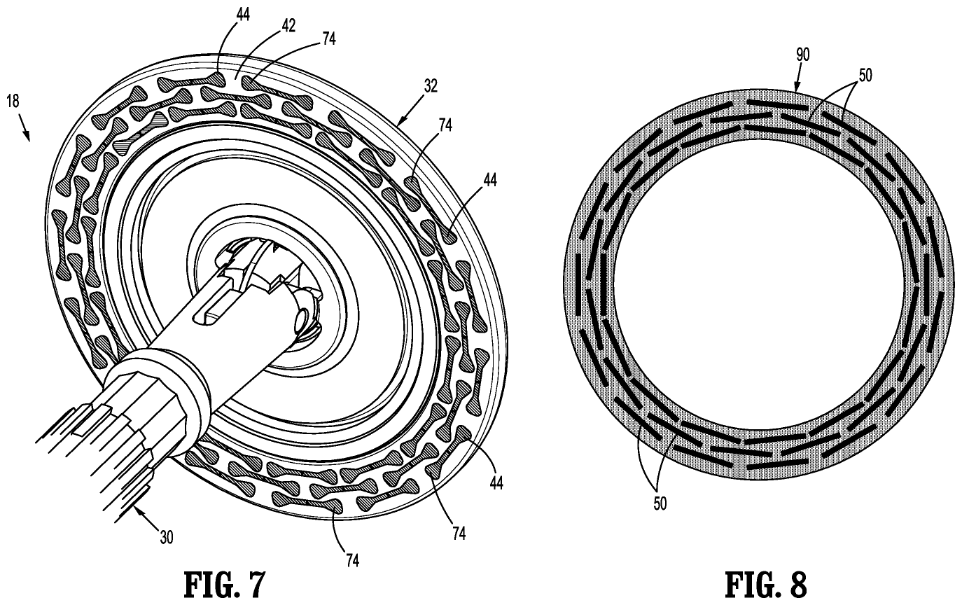

FIGS. 5-8 illustrate distal portion of the stapling device 10 (FIG. 1) during an anastomosis procedure with tissue clamped between the staple cartridge 61 and the anvil surface 42 of the anvil head 32 in optimal fashion. As used herein, “optimal fashion” means the muscularis and submucosal tissue from both ends 80a and 82a of the first and second vessel tracts 80 and 82 are clamped between staple cartridge 61 and the anvil surface 42 of the anvil head 32. When the ends 80a and 82a of the first and second vessel tracts 80 and 82 are clamped between the staple cartridge 61 and the anvil surface 42 in optimal fashion and the stapling device 10 (FIG. 1) is fired to eject the staples 50 through both ends 80 and 82, the resistance exerted on the staple legs 50a staples 50 as the staple legs 50a pass through ends 80a and 82a will cause the staple legs 50a to take a shallow trajectory within the staple forming pockets 44 such that the staple legs 50a will penetrate only the first and/or second layers 70 and 72 of colored coating material within the staple forming pockets 44 of the anvil surface 42. This penetration will remove a portion of the layers 70 and 72 of material to expose the underlying layer, e.g., the third layer 72, which has a predetermined color, e.g., green.

After the anastomosis procedure, a clinician can remove the stapling device 10 from the vessel “V” of the patient and inspect the anvil surface 42. If the exposed portions of the anvil pockets 44 within the staple forming pockets 44 are a uniform color, e.g., yellow or green, this provides an indication to a clinician that the ends 80a and 80b of the first and second vessel sections 80 and 82 were clamped in optimal fashion and that there is a high probability the anastomotic donut 90 is sound and no leakage exists at the anastomotic site. In such an instance, a clinician may decide that further inspection of the anastomotic donut 90 is not warranted.

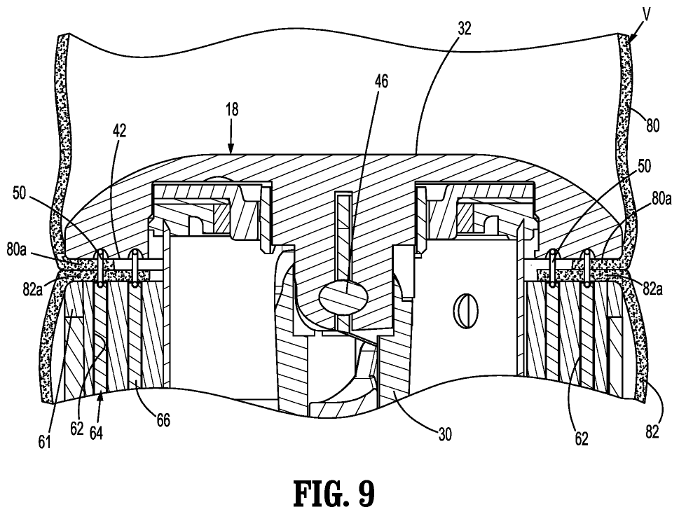

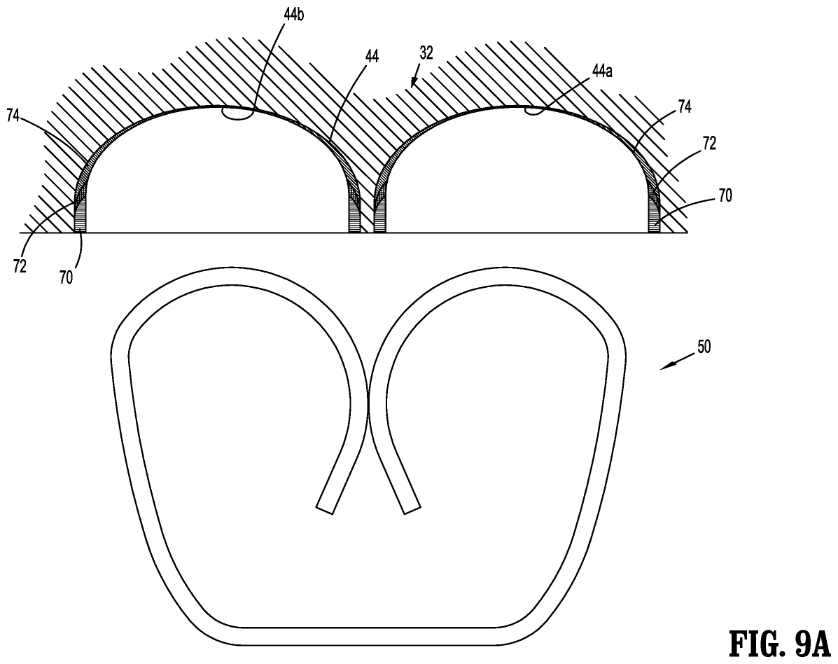

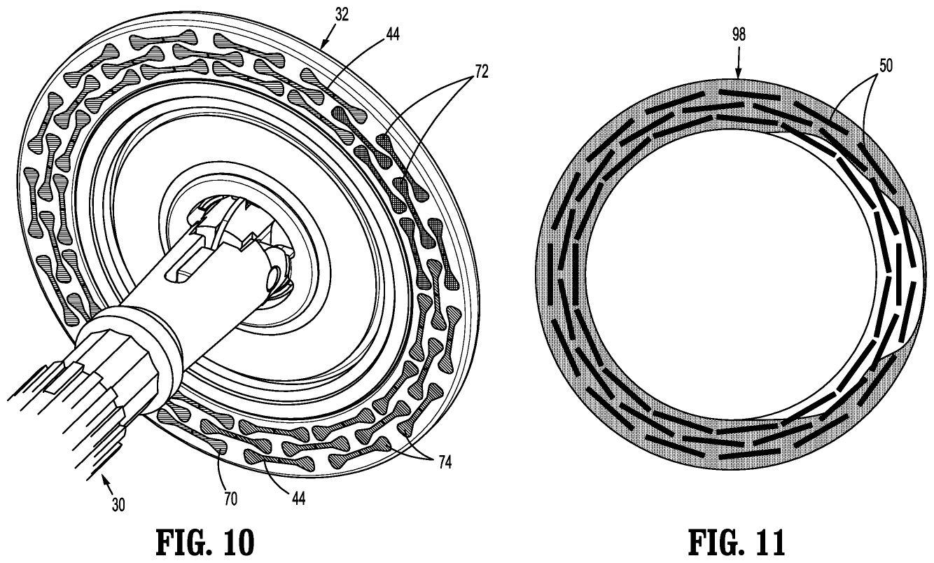

FIGS. 9-11 illustrate the distal portion of the stapling device 10 (FIG. 1) during an anastomosis procedure with tissue clamped between the staple cartridge 61 and the anvil surface 42 of the anvil head 32 in sub-optimal fashion. As used herein, “sub-optimal fashion” means the muscularis and submucosal tissue from both ends 80a and 82a of the first and second vessel tracts 80 and 82 has not been clamped between staple cartridge 61 and the anvil surface 42 of the anvil head 32. When the ends 80a and 82a of the first and second vessel tracts 80 and 82 are clamped between the staple cartridge 61 and the anvil surface 42 in sub-optimal fashion and the stapling device 10 (FIG. 1) is fired to eject the staples 50 through both ends 80a and 82a, the limited resistance exerted on the staple legs 50a of the staples 50 will allow the staple legs 50a to take a deep trajectory within the staple forming pockets 44 such that the staple legs 50a will penetrate all the layers 70, 72, and 74 of colored coating material within the staple pockets 44 of the anvil surface 42. This penetration will remove the layers 70, 72, and 74 of material to expose the underlying layer, e.g., the lowermost layer 74 and/or the surface of the anvil surface 42 beneath the layers 70, 72, and 74 of colored coating material, which has a predetermined color, e.g., green or the color of the material forming the anvil head, e.g., stainless steel.

After the anastomosis procedure, a clinician can remove the stapling device 10 from the vessel “V” of the patient and inspect the anvil surface 42. If the anvil pockets 44 show the color of the third layer 74 of the colored coating material or the color of the anvil surface of the anvil head 32, this provides an indication to a clinician that the ends 80a and 80b of the first and second vessel sections 80 and 82 were clamped in sub-optimal fashion and the there is a probability that the anastomotic donut 98 (FIG. 11) is not sound. It is also noted that the exposed color of the anvil surface 42 within the staple forming pockets 44 should be substantially uniform about the anvil surface 42. If the depth of penetration of the staples 50 into the staple forming pockets 44 is not substantially uniform as indicated by the exposed colors of the layers 70, 72, and/or 74 of colored materials about the anvil surface 42, this provides an indication to the clinician that there is a higher probability that the anastomotic donut 98 is not sound. In such an instance, the clinician may decide that further inspection is warranted and conduct further inspection of the anastomotic donut 98 (FIG. 11) using traditional techniques, e.g., with the assistance of a colonoscope.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. An anvil assembly comprising:
an anvil shaft having a proximal portion and a distal portion;
an anvil head having an anvil surface defining staple forming pockets; and
a plurality of layers of coating material positioned on the anvil surface within the staple forming pockets, the plurality of layers of coating material including an inner layer having a first color and an outer layer having a second color that is different from the first color, the outer layer positioned over the inner layer.

2. The anvil assembly of claim 1, wherein the plurality of layers of coating material includes a middle layer having a third color that is different than the first and second colors, the middle layer positioned between the inner and outer layers.

3. The anvil assembly of claim 1, wherein the plurality of layers of coating material are formed from color coated metals.

4. The anvil assembly of claim 1, wherein the anvil head includes a central post that is secured to the proximal portion of the anvil shaft by a pivot member.

5. The anvil assembly of claim 1, wherein the proximal portion of the anvil shaft is configured to be releasably coupled to a surgical stapling device.

6. The anvil assembly of claim 1, wherein the plurality of layers of coating material are applied to the anvil surface within the staple forming pockets by vapor deposition.

7. The anvil assembly of claim 2, wherein inner layer has a thickness of from about 0.3 mm to about 0.5 mm, the middle layer has a thickness of from about 0.3 mm to about 0.5 mm, and outer layer has a thickness of from about 0.3 mm to about 0.5 mm.

8. The anvil assembly of claim 1, wherein each of the staple forming pockets includes first and second pocket portions and a linear portion, the linear portion extending between and interconnecting the first and second pocket portions.

9. A surgical stapling device comprising:
an elongate body having a proximal portion and a distal portion;
a reload assembly supported on the distal portion of the elongate body, the reload assembly including a shell housing, a staple cartridge, staples supported within the staple cartridge, and a pusher, the pusher being movable within the shell housing from a retracted position to an advanced position to eject the staples from the staple cartridge; and
an anvil assembly including:
an anvil shaft having a proximal portion and a distal portion;
an anvil head having an anvil surface defining staple forming pockets; and
a plurality of layers of coating material positioned on the anvil surface within the staple forming pockets, the plurality of layers of coating material including an inner layer having a first color and an outer layer having a second color that is different from the first color, the outer layer positioned over the inner layer.

10. The surgical stapling device of claim 9, wherein the reload assembly includes a cutting blade supported on the pusher.

11. The surgical stapling device of claim 9, wherein the plurality of layers of coating material includes a middle layer having a third color that is different than the first and second colors, the middle layer positioned between the inner and outer layers.

12. The surgical stapling device of claim 9, wherein the plurality of layers of coating material are formed from color coated metals.

13. The surgical stapling device of claim 9, wherein the anvil head includes a central post that is secured to the proximal portion of the anvil shaft by a pivot member.

14. The surgical stapling device of claim 9, wherein the proximal portion of the anvil shaft is configured to be releasably coupled to a surgical stapling device.

15. The surgical stapling device of claim 9, wherein the plurality of layers of coating material are applied to the anvil surface within the staple forming pockets by vapor deposition.

16. The surgical stapling device of claim 11, wherein inner layer has a thickness of from about 0.3 mm to about 0.5 mm, the middle layer has a thickness of from about 0.3 mm to about 0.5 mm, and outer layer has a thickness of from about 0.3 mm to about 0.5 mm.

17. The surgical stapling device of claim 9, further including a handle assembly, the handle assembly being coupled to the proximal portion of the elongate body.

18. The surgical stapling device of claim 9, wherein each of the staple forming pockets includes first and second pocket portions and a linear portion, the linear portion extending between and interconnecting the first and second pocket portions.

19. An anvil assembly comprising:
an anvil shaft having a proximal portion and a distal portion;
an anvil head having an anvil surface defining staple forming pockets; and
a plurality of layers of coating material positioned on the anvil surface within the staple forming pockets, each of the plurality of layers of coating material formed of color coated metal, the plurality of layers of coating material including an inner layer having a first color, a middle layer having a second color, and an outer layer having a third color, each of the first, second, and third colors being different from each other.

20. The anvil assembly of claim 19, wherein the anvil head includes a central post that is secured to the proximal portion of the anvil shaft by a pivot member.
